(19) United States
(12) Patent Application Publication
(10) Pub. No.: US 2013/0344474 A1
(43) Pub. Date: Dec. 26, 2013

(54) METHOD AND KIT FOR DETECTION OF CHRONIC HEPATITIS B

(71) Applicants: FUJIREBIO INC., Tokyo (JP); NATIONAL HOSPITAL ORGANIZATION, Tokyo (JP)

(72) Inventors: Hiroshi YATSUHASHI, Omura-shi (JP); Atsushi KANEKO, Tokyo (JP); Chiaki TAKAHASHI, Tokyo (JP)

(73) Assignees: NATIONAL HOSPITAL ORGANIZATION, Tokyo (JP); FUJIREBIO INC., Tokyo (JP)

(21) Appl. No.: 13/725,709

(22) Filed: Dec. 21, 2012

(30) Foreign Application Priority Data
Jun. 20, 2012 (JP) 2012-139189

(51) Int. Cl. G01N 33/68

(52) U.S. Cl. CPC G01N 33/6854
USPC 435/5

(57) ABSTRACT

The novel means by which whether a subject has chronic hepatitis B or not can be determined easily with a high degree of accuracy is disclosed. The method for detection of chronic hepatitis B according to the present invention comprises measuring the amount of anti-HBc IgG in a sample separated from a subject. In this method, the measured amount of anti-HBc IgG which is not less than a predetermined cut-off value is indicative of chronic hepatitis B in the subject. The cut-off value may be a value not less than 40 IU/mL, e.g., a value selected from the range of 100 IU/mL to 200 IU/mL. By the present invention, chronic hepatitis B can be detected based on the amount of anti-HBc IgG without measuring the total amount of HBc antibodies including anti-HBc IgM.

### RELATED APPLICATIONS

This application claims the priority of Japanese Patent Application No. 2012-139189 filed Jun. 20, 2012, the entire contents of which are hereby incorporated by reference into the present application.

### FIELD OF THE INVENTION

The present invention relates to a method and a kit for detection of chronic hepatitis B.

### BACKGROUND OF THE INVENTION

Hepatitis B, the most common hepatic disease at present, is a viral hepatitis caused by infection of hepatitis B virus (HBV) to humans.

The main body of the hepatitis B virus is a spherical particle having a double-shelled structure with a diameter of 42 nm, called “Dane particle”. It is known that Dane particles are composed of the HBs antigen which covers the surface of the particles and a central core structure with a diameter of 27 nm which comprises HBc antigen (core antigen), HBe antigen, and double-stranded circular DNA encoding virus genes.

These antigens and autoantibodies against them have been targeted for diagnosis of virus infection. In particular, an antibody against HBc antigen (hereinafter referred to as “anti-HBc antibody”) is used as a maker which can detect HBV infection widely, because it is detected early after HBV infection and continues to be detected for a long time after infection. Anti-HBc antibody includes isotype IgM and isotype IgG. Anti-HBc IgM is detected transiently at a high titer for a period of about 3 to 12 months in the early phase of HBV infection in patients with acute hepatitis B, whereas, anti-HBc IgG continues to be detected positively for a long period of time after HBV infection.

In diagnosis of hepatitis B it is important to discriminate between acute hepatitis B and chronic hepatitis B, because they are different from each other in the clinical course as well as the prognosis. Generally the titers of the total amount of anti-HBc antibodies including anti-HBc IgM and anti-HBc IgG (hereinafter referred to as “HBc total antibody”) are low in cases of acute hepatitis B and high in cases of chronic hepatitis B. Therefore, discrimination between acute hepatitis B and chronic hepatitis B is commonly carried out based on the titers of the HBc total antibody. Although such a procedure enables to discriminate between acute and chronic hepatitis B to some extent, it is demanded to further improve the diagnostic accuracy.

Since anti-HBc IgM is detected transiently at a high titer in the early phase of HBV infection in patients with acute hepatitis B as described above, clinical diagnosis of acute hepatitis B is carried out by measuring anti-HBc IgM (Rumiko NAKAO et al., Japanese Journal of Medicine and Pharmaceutical Science, 52(5):847-858, 2004). However, this method cannot distinguish between healthy individuals and chronic hepatitis B patients, and therefore it is necessary to carry out measurement of HBs antigen and/or HBc total antibody in addition to anti-HBc IgM in order to diagnose chronic hepatitis B. Thus, a method for diagnosing chronic hepatitis B by discriminating between acute hepatitis B and chronic hepatitis B more simply is demanded.

### SUMMARY OF THE INVENTION

Accordingly, an object of the present invention is to provide a method by which determination of chronic hepatitis B can be carried out easily with a high degree of accuracy.

The present inventors intensively studied on hepatitis B virus infection to find, surprisingly, that chronic hepatitis B can be detected distinctly from acute hepatitis B with a good accuracy by measuring anti-HBc IgG in a sample, thereby completing the present invention.

That is, the present invention provides a method for detection of chronic hepatitis B, comprising measuring the amount of anti-HBc IgG in a sample separated from a subject, wherein the measured amount of anti-HBc IgG which is not less than a predetermined cut-off value is indicative of chronic hepatitis B in said subject. The present invention also provides a kit for detection of chronic hepatitis B, which is for carrying out the above-described method of the present invention, said kit comprising HBc antigen and an anti-IgG antibody or antigen-binding fragment thereof.

By the present invention, a method by which determination of chronic hepatitis B can be carried out easily with a high degree of accuracy is provided. Using the method according to the present invention, chronic hepatitis B can be easily diagnosed distinctly from acute hepatitis B with a high degree of accuracy.

### BRIEF DESCRIPTION OF THE DRAWINGS

FIG. 1 shows the measured amounts of anti-HBc IgG in samples from individuals without hepatitis B, patients with acute hepatitis B and patients with chronic hepatitis B.

### DETAILED DESCRIPTION OF THE INVENTION

It should be understood that this invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise.

In the present invention, the amount of anti-HBc IgG in a sample separated from a subject is measured, and the measured amount of anti-HBc IgG is compared to a prescribed cut-off value, thereby detecting chronic hepatitis B. As shown in the Examples described below, the concentration of anti-HBc IgG in samples derived from patients with hepatitis B is higher than the concentration of anti-HBc IgG in samples derived from individuals without hepatitis B; and moreover, the concentration of anti-HBc IgG in samples from patients with chronic hepatitis B is higher than the concentration of anti-HBc IgG in samples from patients with acute hepatitis B. Thus, by setting the amount of anti-HBc IgG by which a group of patients with chronic hepatitis B and a group of patients with acute hepatitis B can be distinguished with a sufficient accuracy as a cut-off value, whether a subject has chronic hepatitis B or not can be determined based on whether the measured amount of anti-HBc IgG in a sample is not less than the cut-off value or not. According to the present invention, chronic hepatitis B can be detected based on the amount of anti-HBc IgG without measuring anti-HBc IgM nor HBc total antibody in which anti-HBc IgM is included.

A method per se for measuring an antibody is a well-known, conventional procedure. In the present invention, any method may be used as long as anti-HBc IgG can be specifically detected thereby. For example, various immunoassays including, but not limited thereto, chemiluminescent immunoassay (CLIA), chemiluminescent enzyme immunoassay (CLEIA), radioimmunoassay (RIA), ELISA and fluoroimmunoassay may be used.

For example, when anti-HBc IgG in a sample is measured by 2-step sandwich CLEIA, HBc antigen immobilized on a solid-phase support is used. The immobilized HBc antigen is brought into contact with anti-HBc antibodies contained in a sample to react HBc antigen with anti-HBc antibodies, so that anti-HBc antibodies including anti-HBc IgG are bound to the support through HBc antigen molecules. After the reaction, B/F separation and washing are carried out. Next, a labeled anti-IgG antibody is added to the system, and the anti-HBc IgG bound to the HBc antigen is allowed to react with the labeled antibody, followed by B/F separation and washing to remove unreacted labeled anti-IgG antibody molecules. Thereafter, signals from the labeled anti-IgG antibody bound to the support through HBc antigen and anti-HBc IgG are detected by an appropriate method, so that anti-HBc IgG in the sample can be specifically measured. Although a 2-step method is explained hereinabove, a 1-step immunoassay may also be used in the present invention. The measurement is also carried out using an anti-IgG antibody immobilized on a solid phase and labeled HBc antigen. Competition techniques may also be used to measure anti-HBc IgG.

Any HBc antigen may be used for measurement of anti-HBc IgG as long as it specifically binds to an anti-HBc antibody. Commercially-available recombinant HBc antigen may be used, and recombinant HBc antigen prepared by a well-known genetic engineering technique may also be used. HBc antigen may also be obtained by extraction from hepatitis B virus-infected cells.

As the amino acid sequence of HBc protein and the nucleotide sequence encoding it can be obtained from a known database such as the NCBI GenBank, a skilled person can easily prepare recombinant HBc antigen using a well-known genetic engineering technique. The sequences shown in SEQ ID NOs: 1 and 2 in SEQUENCE LISTING are examples of the known sequence of HBV core gene and HBc protein encoded by it, respectively, and they have been submitted to GenBank under the accession No. AF324125.1. Most of the amino acid sequences of HBc protein which have been ever known are about 95% or more identical to the amino acid sequence shown in SEQ ID NO: 2, or the same as the sequence shown in SEQ ID NO: 2 except that one to several amino acids are substituted, deleted, inserted and/or added, and HBc protein having such an amino acid sequence can be preferably used as HBc antigen for measurement of anti-HBc IgG. However, it should be understood that the sequence of HBc antigen is not restricted thereto, and those having less than 95% identity to the amino acid sequence of SEQ ID NO: 2 or those to which an additional amino acid sequence such as a tag sequence is added to the N-terminus and/or the C-terminus may also be used as HBc antigen as long as those having such a sequence can react with anti-HBc antibodies produced in a body infected by HBV. In the present invention, the term “HBc antigen” includes various kinds of the HBc protein as described above.

The term “identity” of amino acid sequences means a value expressed in percentage which is calculated by aligning two amino acid sequences to be compared such that as many amino acid residues as possible are matched and then dividing the number of the matched amino acid residues by the number of the total amino acid residues. When the above-mentioned alignment is carried out, one or more gaps are appropriately inserted into one or both of the two sequences to be compared as required. Such alignment of sequences can be carried out using a well-known program such as BLAST, FASTA or CLUSTAL W. When one or more gaps are inserted, the above-described number of the total amino acid residues is calculated by counting one gap as one amino acid residue. When the thus counted numbers of the total amino acid residues are different between the two sequences to be compared, the identity (%) is calculated by dividing the number of the matched amino acid residues by the number of the total amino acid residues of the longer sequence. In the case where the amino acid sequence to be compared comprises any other arbitrary sequence(s) (e.g. tag sequences, linker sequences, other protein sequences, etc.) at one or both ends, the identity should be calculated ignoring such an arbitrary sequence(s).

The 20 types of amino acids which constitute the naturally-occurring proteins may be classified into groups; for example, neutral amino acids with side chains having low polarity (Gly, Ile, Val, Leu, Ala, Met, Pro), neutral amino acids having hydrophilic side chains (Asn, Gln, Thr, Ser, Tyr, Cys), acidic amino acids (Asp, Glu), basic amino acids (Arg, Lys, His) and aromatic amino acids (Phe, Tyr, Trp). Amino acids classified in the same group have a similar property, and it is known that, in most cases, substitution of one or more amino acids within the same group does not change the properties of the protein. Therefore, a protein whose amino acid sequence is partially different from the known HBc amino acid sequence shown in SEQ ID NO: 2 as one example is highly likely to maintain the binding property to anti-HBc antibodies if the difference is due to substitution of one or more amino acids within the same group.

A solid-phase support is not restricted and may be a support commonly used in a conventional immunoassay. For example, plastic plates, fine particles, fibrous materials and the like may be used as a solid-phase support. Conventionally known materials may be used for these supports. Examples of the fine particles include glass beads, various plastic beads such as polystyrene beads, latex particles, various ferrite particles (see, e.g. JP 03-115862 A) and the like. Examples of the fibrous materials include those composed of cellulose, nitrocellulose, chitosan, polyethylene glycol polymer, silane polymer or the like.

Immobilization of HBc antigen on the above-described supports may be carried out by appropriately using a known technique such as physical adsorption, chemical binding using a cross-linker or the like.

As for the anti-IgG antibody used in the present invention, any antibody may be used as long as it specifically binds to IgG, and an anti-IgG antibody or antigen-binding fragment thereof may be used. The anti-IgG antibody to be used may be preferably selected depending on the origin of the sample. For example, when a sample from human is used, it is preferred to use an anti-human IgG antibody or antigen-binding fragment thereof. Specific examples of the anti-IgG antibody or antigen-binding fragment thereof include a polyclonal antibody obtained by immunization of an experimental animal such as mouse, rabbit, chicken, goat, sheep or bovine with IgG (e.g. human IgG in the case of preparation of anti-human IgG or antigen-binding fragment thereof); a monoclonal antibody produced by hybridoma cells obtained by separating spleen cells from an immunized experimental animal and then fusing the cells with myeloma cells; a monoclonal antibody produced by cells prepared by immortalizing spleen cells or blood leukocytes separated from an immunized experimental animal by EB virus; a recombinant antibody; a fragment of any of the above-described antibodies having a binding property to IgG (e.g., Fab, Fab′, F(ab′)₂, Fv, scFv) and the like. Any molecule may be used as long as it has a high specificity and avidity to IgG. The anti-IgG antibody or antigen-binding fragment thereof used in the present invention may be a commercially available product, or may be one prepared by a well-known, conventional method.

Examples of the labeling substance used for labeling of an anti-IgG antibody or antigen-binding fragment thereof or HBc antigen include enzymes, fluorescent substances, chemiluminescent substances, chromogenic substance, radioactive substances and the like. As an enzyme, known enzymes such as, but not limited to, alkaline phosphatase (ALP), peroxidase, β-galactosidase and the like may be used. It is desirable to use ALP in order to provide a measurement system with a high detection sensitivity.

In the case where an enzyme is used as a labeling substance, a substrate appropriately selected depending on the kind of the enzyme used, such as chromogenic substrate, fluorescent substrate or luminescent substrate, may be added to the measurement system, and signals generated by the enzymatic reaction such as coloring or luminescence may be measured with an absorptiometer, a luminometer or the like. For example, when ALP is used as a labeling substance, a luminescent substrate such as disodium 3-(4-methoxyspiro(1,2-dioxetane-3,2′-tricyclo[3.3.1.13,7]decan)-4-yl)phenyl phosphate (e.g. tradename AMPPD (registered trademark)) or the like may be used. When biotin or hapten is used as a labeling substance, streptavidin or an anti-hapten antibody to which an enzyme, fluorescent substance, chemiluminescent substance, chromogenic substance, a radioactive substance or the like is bound may be used.

As for the labeling of an anti-IgG antibody, the antibody may be directly labeled by a known covalent binding method or the like, or may be indirectly labeled using any other binding pair such as biotin-avidin or the like.

Anti-HBc IgG in a subject sample may be quantified by carrying out measurement of anti-HBc IgG in a plurality of standard samples each containing a known concentration of anti-HBc IgG in the manner as described above to measure the signal levels; plotting the correlation between the measured signal levels and the known concentrations of anti-HBc IgG in the standard samples to obtain a standard curve; carrying out the same operation on the subject sample which contains an unknown concentration of anti-HBc IgG to measure the signal level; and comparing the measured signal level of the subject sample with the standard curve to calculate the amount of anti-HBc IgG in the subject sample. The antibody amount can be expressed in IU/mL (international unit/mL) by preparing a standard curve using the WHO First International Standard for anti-HBc antibody (WHO International Standard: First International Standard for anti-Hepatitis B core antigen (anti-HBc), plasma, human. NIBSC code: 95/522), and then calculating the amount of anti-HBc IgG in a sample separated from a subject referring to the thus prepared standard curve. The amount of the antibody in IU/mL can also be calculated by using any other reference standard which has been calibrated against the above-mentioned international standard.

In the method of the present invention, the amount of anti-HBc IgG in a sample is measured in the manner as described above, and thereafter the measured amount is compared to a predetermined cut-off value. The predetermined cut-off value is an indicator of whether a subject has chronic hepatitis B or not, and it can be determined that a subject has chronic hepatitis B in the case where the measured amount of anti-HBc IgG is not less than the cut-off value. That means, chronic hepatitis B is detected in the case where the measured amount of anti-HBc IgG is not less than the predetermined cut-off value.

The cut-off value, an indicator of chronic hepatitis B, may be determined as follows. Immunoassay of anti-HBc IgG is carried out on a plurality of samples (it is desirable to use as many samples as possible) from a group of patients known to have acute hepatitis B and a group of patients known to have chronic hepatitis B, and the measured levels of the signal are applied to a standard curve prepared in the above-described manner to calculate the amount of anti-HBc IgG in each sample, thereby obtaining the measured amount of anti-HBc IgG. Based on the obtained measured amounts, a value by which the group of patients with acute hepatitis B and the group of patients with chronic hepatitis B can be distinguished with a sufficient diagnostic accuracy is determined. The thus determined value can be used as a cut-off value which serves as an indicator of chronic hepatitis B.

When a standard curve is prepared using the WHO First International Standard for anti-HBc antibody or any other reference standard calibrated against the international standard and the amount of antibody is calculated using the thus prepared standard curve, a cut-off value expressed in IU/mL can be obtained. Such an cut-off value expressed in the international unit can be commonly used in the world, regardless of the types of the measurement system (reagents, apparatuses) used or the place where the method is carried out.

The cut-off value may also be expressed as a relative value (Cut Off Index; C.O.I.), which is calculated by a formula specifically established for each antibody measurement system. Similarly to the case where the cut-off value is calculated as an antibody concentration, the relative value C.O.I. may also be calculated as follows. That is, anti-HBc IgG is measured in a plurality of samples from a group of patients known to have acute hepatitis B and a group of patients known to have chronic hepatitis B, and C.O.I. is calculated using the formula established for the measurement system used. Thereafter, a value (C.O.I.) by which the group of patients with acute hepatitis B and the group of patients with chronic hepatitis B can be distinguished with a sufficient diagnostic accuracy is obtained, and the obtained value can be used as a cut-off value.

Here it is assumed that a certain value is determined as a cut-off value. Referring to an individual whose anti-HBc IgG level is not less than the cut-off value as “chronic hepatitis B positive” and an individual whose anti-HBc IgG level is less than the cut-off value as “acute hepatitis B positive”, the diagnostic accuracy at the said cut-off value may be calculated by the following formula:

([Total number of individuals having acute hepatitis B]−[Number of acute hepatitis B positive]+[Number of chronic hepatitis B positive])/([Total number of individuals having acute hepatitis B]+[Total number of individuals having chronic hepatitis B]).

In the present invention, the cut-off value may be determined so that the diagnostic accuracy for the discrimination between acute hepatitis B and chronic hepatitis B is 80% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more, or is 100%.

Specifically, the cut-off value used in the present invention may be a value of not less than 40 IU/mL. The cut-off value is preferably a value of between 40 IU/mL and 470 IU/mL; more preferably a value of between 80 IU/mL and 320 IU/mL; still more preferably a value of between 100 IU/mL and 200 IU/mL. In the case where the cut-off value is selected from the range of 40 IU/mL to 470 IU/mL, chronic hepatitis B can be discriminated from acute hepatitis B with a diagnostic accuracy of not less than 85.0% (see, Examples below). In the case where the cut-off value is selected from the range of 80 IU/mL to 320 IU/mL or the range of 100 IU/mL to 200 IU/mL, the discrimination between acute and chronic hepatitis B can be achieved with a still higher diagnostic accuracy of not less than 90.0% or not less than 93.8%, respectively. Thus, by comparing the measured amount with the cut-off value as described above, chronic hepatitis B can be detected distinctly from acute hepatitis B with a high degree of accuracy.

In the present invention, the second cut-off value for discrimination between healthy individuals and hepatitis B patients (specifically acute hepatitis B patients) may be used. Hereinafter the previously-described cut-off value which serves as an indicator of whether a subject has chronic hepatitis B or not is referred to as “the first cut-off value” in contrast with the second cut-off value. In a similar manner to the first cut-off value, the second cut-off value may be determined, for example, as follows. Immunoassay of anti-HBc IgG is carried out on a plurality of samples (it is desirable to use as many samples as possible) from a group of patients known to have acute hepatitis B and a group of individuals known not to have hepatitis B, and the measured signal levels are compared with a standard curve prepared in the above-described manner to calculate the amount of anti-HBc IgG in each sample, thereby obtaining the measured amount of anti-HBc IgG. Based on the obtained measured amounts, a value by which the group of healthy individuals and the group of patients with acute hepatitis B can be distinguished with a sufficient diagnostic accuracy is determined. The thus determined value can be used as an indicator of whether a subject is healthy or has chronic hepatitis B (more specifically, acute hepatitis B).

Similarly to the first cut-off value, the second cut-off value may also be expressed in antibody concentration (IU/mL) using the international unit or in C.O.I.

The second cut-off value may be determined so that the diagnostic accuracy for discrimination between healthy individuals and acute hepatitis B patients is 80% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more, or is 100%. Specifically, the second cut-off value may be a value selected from the range of 0.7 IU/mL to 0.9 IU/mL, but not limited thereto. In this case, the diagnostic accuracy may be calculated by the following formula:

([Total number of healthy individuals]−[Number of healthy positive]+[Number of acute hepatitis B positive])/([Total number of healthy individuals]+[Total number of individuals having acute hepatitis B])

As used herein, the terms “healthy individual” and “individual without hepatitis B” refer to an individual who is not infected by HBV and is not suffering from hepatitis B. A healthy individual with no history of hepatitis B usually tests negative for various HBV proteins and anti-HBV protein antibodies including HBs antigen and anti-HBs antibodies. When determining the second cut-off value, a plurality of samples from healthy individuals or individuals without hepatitis B are used, and the fact that individuals from whom the samples are derived are healthy or without hepatitis B may be judged according to the clinical symptoms and various blood test findings. For example, as shown in the Examples below, a sample from an individual who shows no hepatitis symptoms and tests negative for both HBs antigen and anti-HBs antibodies may be used as a sample from a healthy individual or a sample from an individual without hepatitis B when determining the second cut-off value.

The first and the second cut-off values may be determined based on the diagnostic accuracy as described above. However, the procedure for determination is not limited thereto, and both cut-off values may also be determined appropriately based on any other index commonly used in in-vitro diagnostic reagents such as sensitivity, specificity or the like.

It is not necessary to determine the first and the second cut-off values each time the present invention is carried out, and the cut-off values once determined using a certain measurement system can be used as they are when carrying out the present invention later as long as the same measurement system is used for measurement of anti-HBc IgG. In the case where the cut-off values are expressed in the international unit, the same cut-off values can be used regardless of the measurement system used. In the present invention, for example, the above-described preferred cut-off values expressed in the antibody concentration using the international unit can be used as they are.

Examples of the sample used in the present invention include biological body fluids such as whole blood, plasma, serum, urine, saliva, cerebrospinal fluid and the like and liver tissues, and preferred examples of the sample include whole blood, serum and plasma samples, but the samples are not limited thereto.

The subject is not restricted as long as it is a mammal, and is preferably human. The subject may be a patient suspected of having hepatitis B or a patient who has developed hepatitis (especially hepatitis B). For example, in the case where the present invention is carried out on a sample separated from a patient who has developed hepatitis, the amount of anti-HBc IgG in the sample is supposed to be less than the first cut-off value if the patient is suffering from acute hepatitis B or hepatitis other than hepatitis B. Therefore, chronic hepatitis B can also be detected by the method of the present invention even in such a case.

When the method of the present invention is carried out on a patient who has developed or is suspected of having hepatitis B, whether the patient has acute hepatitis B or chronic hepatitis B can be determined using only the first cut-off value. Specifically, the patient can be diagnosed to have chronic hepatitis B if the amount of anti-HBc IgG in the sample is not less than the first cut-off value, and diagnosed to have acute hepatitis B if the amount of anti-HBc IgG in the sample is less than the first cut-off value.

In the case where the second cut-off value is used in combination with the first cut-off value, it may be possible to distinguish among the three parties of healthy (mainly with no history of hepatitis B), acute hepatitis B and chronic hepatitis B.

The present invention also provides a kit for detection of chronic hepatitis B by the method of the present invention described above. The kit comprises HBc antigen and an anti-IgG antibody or antigen-binding fragment thereof, and using this kit anti-HBc IgG in a sample is measured by an immunoassay. Conditions of the immunoassay, HBc antigen and anti-IgG antibody or antigen-binding fragment thereof are the same as described above. For example, HBc antigen may be bound to a solid support such as a plate or particles (e.g. magnetic particles, latex particles and the like), and an anti-IgG antibody or antigen-binding fragment thereof may be labeled with an arbitrary labeling substance. Alternatively, an anti-IgG antibody or antigen binding fragment thereof may be bound to a solid support, and HBc antigen may be labeled.

The detection kit for chronic hepatitis B may further comprise written instructions in which the first cut-off value which serves as an indicator of chronic hepatitis B is described. In the written instructions, the second cut-off value which serves as an indicator of whether a subject is healthy or has acute hepatitis B may also be described. Since anti-HBc IgM does not have to be measured in the method of the present invention, the detection kit of the present invention does not have to comprise an anti-IgM antibody nor an antigen-binding fragment thereof. The kit may further comprise other reagents commonly contained in immunoassay kits such as a substrate solution appropriately selected depending on the labeling substance used, a diluent for samples, a washing buffer and the like.

### EXAMPLES

The present invention will now be described more concretely by way of examples thereof. However, the present invention is not restricted to the examples below.

### Example

### Discrimination of hepatitis B by Measurement of Anti-HBc IgG

### (1) Preparation of HBc Antigen-Binding Particles

Particles to which recombinant HBc antigen is bound were prepared as follows.

To 1 mg of magnetic ferrite particles (produced by NIPPON PAINT), 1.5 of recombinant HBc antigen (produced by FUJIREBIO INC.) was added, and the mixture was incubated at 25° C. for 1 hour with gentle stirring. After the reaction, ferrite particles were collected by a magnet to separate them from the reaction solution, and the particles were washed with Buffer A (50 mM Tris buffer pH7.2, 0.15% NaCl, 0.1% sodium azide, 2% BSA), thereby obtaining recombinant HBc antigen-binding particles.

### (2) Preparation of ALP-Labeled Anti-IgG Antibody

Mouse anti-human IgG monoclonal antibody (produced by FUJIREBIO INC.) and alkaline phosphatase from calf small intestine (produced by ORIENTAL YEAST CO., LTD.) were conjugated in accordance with Yoshitake et al (J. Biochem. 1982, 92(5), p1413-1424) to prepare ALP-labeled antibody.

Specifically, desalted mouse anti-human IgG monoclonal antibody (final concentration: 3 mg/mL) and pepsin (final concentration: 6 μg/mL) were mixed in 0.1M citrate buffer (pH3.5), and the mixture was allowed to stand at 37° C. for 1 hour to digest the antibody with pepsin. After stopping the reaction, the mixture was purified by gel filtration to obtain an anti-human IgG antibody from which the FC region was removed. Thereafter, 2-mercaptoethanol (final concentration: 10 mM) was added thereto, and the mixture was allowed to stand at 37° C. for 3 hours to carry out thiolation, followed by desalting to obtain a Fab fragment of anti-human IgG antibody.

On the other hand, desalted alkaline phosphatase and N-(4-maleimidobutyryloxy)-succinimide (GMBS) (final concentration: 0.3 mg/mL) were mixed, and the mixture was allowed to stand at 30° C. for 1 hour to carry out maleimidation.

After desalting, the Fab fragment and maleimidated alkaline phosphatase were mixed at a molar ratio of 1:1, and the mixture was allowed to stand at 25° C. for 30 minutes to carry out coupling reaction. To the coupling solution, 2-mercaptoethanol (final concentration: 10 mM) was added, and the mixture was allowed to stand at 4° C. overnight to stop the reaction. After concentration and desalting, 100 mM MES (pH 6.8) containing 1% BSA, 1 mM NaCl and 0.1 mM ZnCl₂ was added to the mixture to adjust the concentration of the antibody to 200 μg/mL, thereby obtaining a solution of ALP-labeled anti-IgG antibody.

### (3) Measurement of Anti-Human HBc IgG

The concentration (IU/ml) of anti-HBc IgG in samples from a patient with chronic hepatitis B was determined using the WHO First International Standard for anti-HBc antibody (WHO International Standard: First International Standard for anti-Hepatitis B core antigen (anti-HBc), plasma, human NIBSC code: 95/522), and the sample was appropriately diluted to obtain standard samples for preparation of a standard curve.

Standard samples were prepared so that they contained anti-HBc antibody (isotype IgG) at concentrations of 0, 1, 20, 50, 100, 200, 400, 600, 800 and 1000 IU/ml, respectively, as calculated based on the above-mentioned WHO First International Standard, and each of the standard samples was 19-fold diluted with Lumipulse (registered trademark) sample diluent (produced by FUJIREBIO INC.). A 10 μL aliquot of each of the diluted standard samples was added to 0.25 mL of the solution of HBc antigen-binding particles prepared in (1), and the mixture was allowed to react at 37° C. for 10 minutes. B/F separation was carried out using a magnet, and after washing the particles, 0.25 mL of the solution of ALP-labeled anti-IgG antibody prepared in (2) was added to the particles, followed by allowing the mixture to react at 37° C. for 10 minutes again. B/F separation was carried out using a magnet, and after washing the particles, 200 μL of Lumipulse (registered trademark) substrate solution (produced by FUJIREBIO INC.) containing a chemiluminescent substrate 3-(2′-spiroadamantane)-4-methoxy-4-(3″-phosphoryloxy)phenyl-1,2-dioxetane disodium salt (AMPPD (registered trademark)) was added to the particles. The mixture was allowed to react at 37° C. for 5 minutes, and luminescence was measured with a luminometer. The fully automated chemiluminescent enzyme immunoassay system (Lumipulse (registered trademark) G1200, produced by FUJIREBIO INC.) was used for the measurement.

A spline curve was obtained from the results of the measurement of the standard samples containing the respective concentrations of the antibody as calculated based on the above-mentioned WHO First International Standard, which was used as a standard curve.

### (4) Measurement of Samples

Samples were measured in accordance with the measurement method described in (3). The subjects studied herein were: 30 cases with a diagnosis of acute hepatitis B, 130 cases with a diagnosis of chronic hepatitis B, and 30 cases without hepatitis B who tested negative for both HBs antigen and anti-HBs antibody. Diagnosis of acute and chronic hepatitis B was carried out in the Nagasaki Medical Center according to the clinical symptoms and various blood test findings. Serum samples from the above-mentioned 190 cases were stored under −20° C., and thawed before measurement.

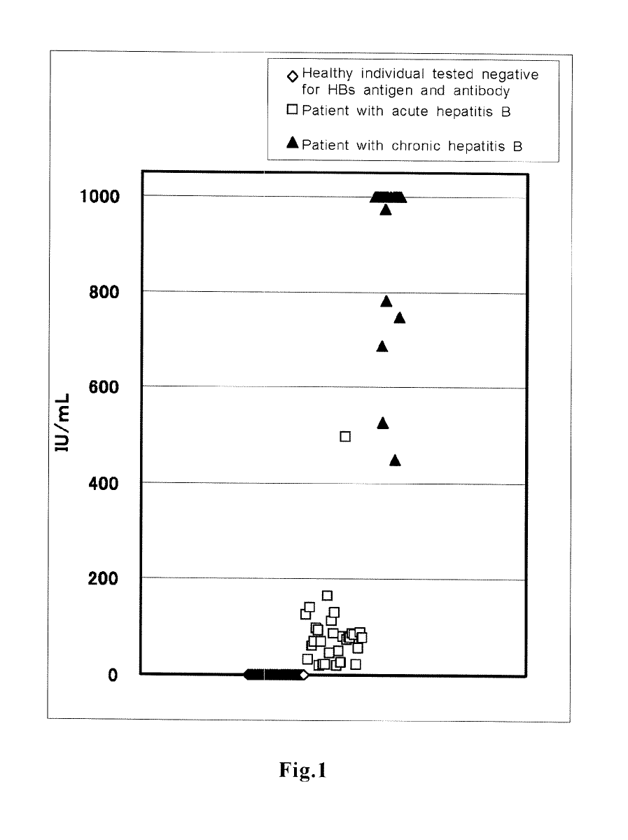

The samples were measured by the method described in (3), and the antibody concentration (IU/mL) of each of the samples were calculated using a standard curve obtained in (3). Results are shown in FIG. 1 and Table 1. In FIG. 1, samples with a concentration of 1000 IU/mL or more were indicated as 1000 IU/mL.

TABLE 1

| Cut-off Value | Acute Hepatitis B | Chronic Hepatitis B | Diagnostic |
|---|---|---|---|
| (IU/mL) | Positive | Positive | Accuracy |
| 30 | 24 | 129 | 84.4% |
| 40 | 23 | 129 | 85.0% |
| 70 | 17 | 127 | 87.5% |
| 80 | 13 | 127 | 90.0% |
| 90 | 8 | 126 | 92.5% |
| 100 | 6 | 126 | 93.8% |
| 110 | 6 | 126 | 93.8% |
| 120 | 5 | 126 | 94.4% |
| 190 | 1 | 122 | 94.4% |
| 200 | 1 | 121 | 93.8% |
| 210 | 1 | 120 | 93.1% |
| 220 | 1 | 119 | 92.5% |
| 250 | 1 | 119 | 92.5% |
| 260 | 1 | 118 | 91.9% |
| 310 | 1 | 117 | 91.3% |
| 320 | 1 | 116 | 90.6% |
| 330 | 1 | 114 | 89.4% |
| 440 | 1 | 108 | 85.6% |
| 450 | 1 | 107 | 85.0% |
| 470 | 1 | 107 | 85.0% |
| 480 | 1 | 106 | 84.4% |

As a result, surprisingly, it was found that the measured amount of anti-HBc IgG in acute patients tended to show a lower value, and that the measured amount of anti-HBc IgG in chronic patients tended to show a higher value.

As shown in FIG. 1 and Table 1, it was revealed that when setting the cut-off value as a value within the range of 40 IU/mL to 470 IU/mL, acute hepatitis B and chronic hepatitis B could be discriminated with a diagnostic accuracy of not less than 85.0%. It was further revealed that the discrimination could be achieved with a much higher diagnostic accuracy when setting the cut-off value as a value within the range of 80 IU/mL to 320 IU/mL or the range of 100 IU/mL to 200 IU/mL; in the former case the diagnostic accuracy was not less than 90.0%, and in the latter case not less than 93.8%.

These results indicate that acute hepatitis B patients and chronic hepatitis B patients can be discriminated with a high degree of accuracy by measuring the concentration of anti-HBc IgG in samples. By the method of the present invention, whether a subject has chronic hepatitis B or not can be determined easily with a high accuracy. Specifically, the concentration of anti-HBc IgG in a sample is measured, and the measured concentration of anti-HBc IgG is compared to a cut-off value by which those having chronic hepatitis B can be discriminated from those having acute hepatitis B and those not having hepatitis B. In the case where the measured antibody concentration is not less than the cut-off value, it can be determined that the sample is from a chronic hepatitis B patient.

Furthermore, as shown in FIG. 1, anti-HBc IgG was hardly detected in samples from individuals without hepatitis B who tested negative for both HBs antigen and anti-HBs antibody. The measured values in these samples were applied to the above-obtained standard curve to find that the concentrations were calculated as 0.4 IU/mL or less. On the other hand, the measured values of anti-HBc IgG in samples from hepatitis B patients were calculated as 18 IU/mL or more. Thus, when setting the second cut-off value as a value within the range of 0.5 IU/mL to 17 IU/mL, e.g., the range of 0.7 IU/mL to 0.9 IU/mL, those not having hepatitis B who tested negative for both HBs antigen and anti-HBs antibody could be discriminated from hepatitis B patients. This result suggests that the above-mentioned second cut-off value of the anti-HBc IgG concentration may be used as an indicator of whether a subject has or does not have hepatitis B.

### Reference Example

### Discrimination of Hepatitis B by Measuring HBc Total Antibody

Acute hepatitis B and chronic hepatitis B were discriminated by a conventional method, in which the total amount of anti-human HBc antibodies including anti-human HBc IgG and anti-human HBc IgM in a sample was measured and discrimination was carried out based on whether the antibody titer is high or low. The HBc total antibody was measured by a competition method using Lumipulse Presto (registered trademark) HBcAb (produced by FUJIREBIO INC.). The same samples as used in (4) above were used in this study.

As a result, discrimination between acute hepatitis B and chronic hepatitis B can be achieved at the highest diagnostic accuracy, i.e., 71.3%, when HBc total antibody was 50 INH %. Thus, it was revealed that, when compared to a conventional discrimination method in which chronic hepatitis B and acute hepatitis B were discriminated based on the total amount of anti-human HBc antibodies, the method of the present invention which was based only on the amount of anti-HBc IgG could discriminate between acute hepatitis B patients and chronic hepatitis B patients with a very high diagnostic accuracy.

### INDUSTRIAL APPLICABILITY

By carrying out the present invention, acute hepatitis B and chronic hepatitis B can be discriminated easily with a high degree of accuracy, thereby determining whether a subject has chronic hepatitis B or not. Thus, the present invention is useful in prevention, diagnosis and treatment of HBV infection.

What is claimed is:

1. A method for detection of chronic hepatitis B, comprising measuring the amount of anti-HBc IgG in a sample separated from a subject, wherein the measured amount of anti-HBc IgG which is not less than a predetermined cut-off value is indicative of chronic hepatitis B in said subject.

2. The method according to claim 1, wherein said cut-off value is a value not less than 40 IU/mL.

3. The method according to claim 2, wherein said cut-off value is selected from the range of 40 IU/mL to 470 IU/mL.

4. The method according to claim 3, wherein said cut-off value is selected from the range of 80 IU/mL to 320 IU/mL.

5. The method according to claim 4, wherein said cut-off value is selected from the range of 100 IU/mL to 200 IU/mL.

6. The method according to claim 1, wherein said subject is human.

7. The method according to claim 1, wherein said subject is a patient suspected of having hepatitis B.

8. The method according to claim 1, wherein said subject is a patient suffering from hepatitis.

9. The method according to claim 1, wherein said sample is blood, serum or plasma.

10. A kit for detection of chronic hepatitis B, which is for carrying out the method according to claim 1, said kit comprising HBc antigen and an anti-IgG antibody or antigen-binding fragment thereof.

11. The kit according to claim 10, comprising neither an anti-IgM antibody nor an antigen-binding fragment thereof.

12. The kit according to claim 10, further comprising written instructions in which the predetermined cut-off value serving as an indicator of chronic hepatitis B is described.


SEQUENCE LISTING (2 sequences)

SEQ ID NO: 1 - DNA, 558 bp, Hepatitis B virus
  CDS (1)..(558)
atggacattgacccttataaagaatttggagctactgtggagttactctcgtttttgccttctgacttctttccttccgtcagagatctcctagacaccgcctcagctctgtatcgagaagccttagagtctcctgagcattgctcacctcaccatactgcactcaggcaagccattctctgctggggggaattgatgactctagctacctgggtgggtaataatttggaagatccagcatccagggatctagtagtcaattatgttaatactaacatgggtttaaagatcaggcaactattgtggtttcatatatcttgccttacttttggaagagagactgtacttgaatatttggtctctttcggagtgtggattcgcactcctccagcctatagaccaccaaatgcccctatcttatcaacacttccggaaactactgttgttagacgacgggaccgaggcaggtcccctagaagaagaactccctcgcctcgcagacgcagatctcaatcgccgcgtcgcagaagatctcaatctcgggaatctcaatgttag

SEQ ID NO: 2 - protein, 185 aa, Hepatitis B virus
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC